(19) United States
(12) Patent Application Publication
(10) Pub. No.: US 2007/0026077 A1
(43) Pub. Date: Feb. 1, 2007

(54) Method of producing dosage units of a solid drug form containing warfarin sodium salt as active component

(76) Inventors: Alas Franc, Brno (CZ); Borek Zaludek, Brno (CZ); Roman Gonec, Brno (CZ); Miroslav Malecek, Brno (CZ); Hana Tkadleckova, Brno (CZ); Anna Petrovicova, Brno (CZ)

Correspondence Address: BUCHANAN, INGERSOLL & ROONEY PC, POST OFFICE BOX 1404, ALEXANDRIA, VA, 22313-1404, US

(21) Appl. No.: 10/575,986

(22) PCT Filed: Oct. 14, 2004
(86) PCT No.: PCT/CZ04/00067

§ 371 Date: Apr. 17, 2006

(30) Foreign Application Priority Data
Oct. 17, 2003 (CZ) PV 2854-03

(51) Int. Cl. A61K 31/724; A61K 31/366; A61K 9/14

(52) U.S. Cl. 424/489; 514/58; 514/457

(57) ABSTRACT

The invention relates to a method of producing dosage units of a solid drug form containing as the active substance warfarin sodium salt in an amount of 1 to 10 mg and having high degree of content uniformity satisfying the Bergum criterion, wherein an aqueous solution of warfarin sodium salt and/or its clathrate which optionally contains in the dissolved state one of the pharmaceutically acceptable excipients co-forming the solid drug form to be prepared but not all the pharmaceutically acceptable excipients co-forming the solid drug form to be prepared, is brought into contact with solid particles of at least one pharmaceutically acceptable excipient co-forming the solid drug form to be prepared, whereupon optionally the particles are dried and optionally mixed with a required amount of solid particles of the remaining pharmaceutically acceptable excipients co-forming the solid drug form to be prepared, and the thus-obtained particulate mixture is formulated into dosage units of the solid drug form.

### FIELD OF THE INVENTION

The invention relates to the method of producing a solid dosage form, containing sodium salt of warfarin as the active component in an amount of 1 to 10 mg and having a high degree of content uniformity meeting the Bergum criterion.

### BACKGROUND OF THE INVENTION

Warfarin is a dicoumarol derivative which antagonizes synthesis of vitamin K dependent coagulation factors (factors VII, IX, X, XII), and thus is utilized as an anticoagulative and antithrombotically active compound. According to recent studies, application of warfarin leads to statistically significant retardation of tumor disease progression and to prolongation of survival time of patients with small-cell lung carcinoma.

On peroral application, warfarin is well absorbed in the gastrointestinal tract, its biological accessibility amounting up to 90%. Maximum plasma concentration of warflrin is achieved in 1 to 9 hours after administration. Higher starting doses of warfarin accelerate the onset of the anticoagulative effect, whereas at doses higher than about 0.75 mg/kg there is no further increase in the rate of the onset of the anticoagulative effect. About 97% of warfarin is bound to plasmatic proteins. In the case of warfarin, the distribution volume amounts to about 0.14 l/kg. Chemically, sodium salt of warfarin is sodium salt of 3-(α-acetonylbenzyl)-4-hydroxy-coumarin of summary formula C₁₉H₁₅NaO₄, of relative molecular weight 330.32 and the following structural formula:

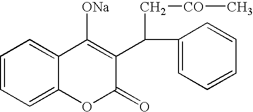

Pharmacologically, warfarin is used in the form of its sodium salt or in the form of clathrate of the sodium salt with isopropanol, the clathrate form being used because of better crystallization of warfarin during its synthesis. From the pharmacological viewpoint there is no difference between both the forms mentioned. Both clathrate of warfarin sodium salt as well as the sodium salt alone are white crystalline light powder which is hygroscopic, well soluble in water and 96% ethanol, soluble in acetone and insoluble in dichloromethane and ether. The synthesis of sodium salt of warfarin and its enantiomers is described in U.S. Pat. No. 5,686,631 and U.S. Pat. No. 5,856,525. Various aspects of application of warfarin sodium salt are described in U.S. Pat. No. 6,056,525, PCT/GB00/WO00/43003 and U.S. Pat. No. 6,559,133. Recently, warfarin sodium salt is provided in the form oftablets of 1 mg, 2 mg, 2.5 mg, 3 mg, 4 mg, 5 mg, 6 mg, 7.5 mg and 10 mg strength.

Solid dosage forms of low content of the active substance must satisfy specific pharmacopoeial requirements concerning content uniformity. The content uniformity represents a critical parameter for drug forms particularly in case of active substances with only narrow interval between their active and toxic doses, which is just the case of warfarin sodium salt. Keeping the content uniformity in individual dosage units of the drug form guarantees that the patient receives only the effective dose of the active substance and not a toxic one. The content uniformity is checked using the Bergum method. In case of meeting the Bergum criterion RSD on 10 to 150 samples of the given drug form batch, this method guarantees on a 95% confidence level that dosage units of the drug form batch pass the content uniformity test.

However, in manufacturing drug forms with a low content of the active compound it is very difficult to reach the high content uniformity limit, particularly in the case when the content of the active compound in a dosage unit does not reach even 1% of the total dosage unit weight. In manufacturing dosage units with low content of the active substance, specific technologies are used such as e.g. mixing in high-speed mixers, milling of active substances with excipients, dispergatory or fluid granulation, and dry granulation. Such procedures often do not lead to the required degree of content uniformity and, moreover, they are laborious and prolonged.

The goal of the invention consists in providing a simple method for obtaining dosage units that contain warfarin sodium salt as the active substance and exhibit a high degree of content uniformity.

### SUMMARY OF THE INVENTION

The present invention relates to a method of producing dosage units of a solid drug form containing warfarin sodium salt as the active substance in an amount of 1 to 10 mg and exhibiting a high degree of content uniformity meeting the Bergum criterion, consisting in bringing into contact a required amount of an aqueous solution of warfarin sodium salt and/or its clathrate, which optionally contains in the dissolved state one of the pharmaceutically acceptable excipients co-forming the solid drug form to be prepared but not all the pharmaceutically acceptable excipients co-forming the solid drug form to be prepared, with solid particles of at least one pharmaceutically acceptable excipient co-forming the solid drug form to be prepared, whereupon optionally the particles are dried and optionally mixed with a required amount of solid particles of the remaining pharmaceutically acceptable excipients co-forming the solid drug form to be prepared, and the obtained particulate mixture is formed into dosage units of the solid drug form.

The bringing into contact of an aqueous solution of warfarin sodium salt and/or its clathrate with solid particles of at least one pharmaceutically acceptable excipient is preferably done by spraying this solution onto these solid particles.

Preferably, an aqueous solution of warfarin sodium salt and/or its clathrate contains 1 to 50% by weight, more preferably 8 to 35% by weight, of warfarin sodium salt and/or its clathrate, based on the weight of the solution.

Beside water, an aqueous solution of warfarin sodium salt and/or its clathrate contains preferably solely warfarin sodium salt and/or its clathrate.

Solid particles of at least one pharmaceutically acceptable excipient, intended for bringing into contact with an aqueous solution of warfarin sodium salt and/or its clathrate, preferably have such particle distribution that the size of at least 90% of these particles is greater than 40 micrometers, the size of at most 10% of these particles is greater than 250 micrometers, and 100% of these particles are of a size not exceeding 300 micrometers.

Pharmaceutically acceptable excipients are preferably selected from a group consisting of a hydrophilic sugar, preferably sucrose, sorbitol, mannitol or lactose, natural or modified starch and cellulose, more preferably a mixture of lactose and microcrystalline cellulose in a weight ratio 10:5 to 11:5.

Solid particles of at least one pharmaceutically acceptable excipient, intended for bringing into contact with aqueous solution of warfarin sodium salt and/or its clathrate, preferably contain added solid particles of pharmaceutically acceptable excipient of a specific surface area of at least 150 m².g⁻¹ in an amount of 0.1 to 2% by weight based on the total weight of solid particles of at least one pharmaceutically acceptable excipient and the said added ingredient intended for bringing into contact with aqueous solution of warfarin sodium salt and/or its clathrate, more preferably added colloidal silicon oxide, in an amount of 0.5% by weight based on the total weight of solid particles of at least one pharmaceutically acceptable excipient and the said added ingredient intended for bringing into contact with aqueous solution of warfarin sodium salt and/or its clathrate.

The mixture of solid or optionally dried particles, obtained after spraying an aqueous solution of warfarin sodium salt and/or its clathrate onto solid particles of at least one pharmaceutically acceptable excipient, is mixed preferably with at least one pharmaceutically acceptable lubricant such as preferably magnesium stearate, zinc stearate, aluminium stearate, colloidal silicon oxide, stearic acid, sodium stearyl fumarate, polyethylene glycol or sodium lauryl sulfate, used in an amount of 0.1 to 10% by weight based on the weight of the obtained mixture, more preferably with magnesium stearate, used in an amount of 1% by weight based on the weight of the obtained mixture.

The mixture of solid or optionally dried particles, obtained after spraying an aqueous solution of warfarin sodium salt and/or its clathrate onto solid particles of at least one pharmaceutically acceptable excipient, is mixed preferably with at least one pharmaceutically acceptable disintegrant such as preferably ultraamylopectin, sodium salt of crosslinked carboxymethylcellulose or crosslinked polyvinylpyrrolidone, used in an amount of 1 to 7% based on the weight of the obtained mixture, more preferably with sodium salt of crosslinked carboxymethylcellulose, used in an amount of 2% by weight based on the weight of the obtained mixture.

With advantage, the obtained particulate mixture is formulated into dosage units of a solid drug form by filling in capsules and/or sachets and/or by pressing to tablets.

The method according to this invention represents an entirely unique technology providing a uniform dispersion of a pharmacologically active substance onto the surface of excipients of a defined particle size distribution. In the course of the manufacturing process according to the invention the surface of the carrier excipients does not change any more, i.e. no granulate is formed, thus eliminating a tedious preparation of binder and also a technologically demanding extrusion and lengthy grinding and sorting by size of the granulate formed. Since in the method of producing the drug form according to the invention the active substance is completely dissolved in water, it is entirely immaterial whether the drug form is prepared from the warfarin sodium salt as such or from its clathrate because during the dissolution of the active substance in water the crystal lattice is disintegrated and the liberated isopropanol is removed during drying of the carrier component with the integrated active substance. In the method according to the invention it is therefore possible to use warfarin sodium salt as well as its clathrate without deteriorating the quality of the final drug form. Spraying of a solution of the active substance onto the surface of the carrier, formed by at least one excipient, may be performed in a fluid processor in air stream, or a solution of the active substance may be sprayed onto the carrier surface in the course of mixing in various types of low-speed or high-speed mixers, or a solution of the active substance may be sprayed onto the carrier surface in a coating drum with subsequent drying. During further handling of the obtained mixture, demixing of the active substance from the carrier may occur as the result of different densities of the active substance and the excipients. Within the framework of the present invention this is suppressed by addition of an excipient of a high specific surface area and a high electrostatic charge. This excipient of a specific surface of at least 150 m².g⁻¹ and of a high electrostatic charge, preferably silicon oxide, prevents demixing of the active substance from the carrier on which it is fixed by means of its surface and electrostatic charge. Therefore, the said excipient is added to the carrier excipients before spraying the active substance solution.

In the following part of the description, the invention is explained in more detail using examples of execution, these examples being only illustrative and not limiting the scope of the invention which is unequivocally defined by the Claims and the Description Part.

### EXAMPLES

### Example 1

In this example, 220 mg tablets were prepared using as the active component clathrate of warfarin sodium salt, theoretically containing 8.34% by weight of isopropanol. The composition of the tablets is given in Table 1 below.

TABLE 1

| | Strength (mg) | | | | |
|---|---|---|---|---|---|
| Component | 1 | 2 | 2.5 | 3 | 4 |
| Clathrate of warfarin | 1.09 | 2.18 | 2.73 | 3.27 | 4.36 |
| sodium salt | | | | | |
| Water | 11.15 | 11.15 | 11.15 | 11.15 | 11.15 |
| Lactose | 144.99 | 143.46 | 146.31 | 142.81 | 141.67 |
| Avicel | 66.00 | 66.00 | 66.00 | 66.00 | 66.00 |
| Aerosil | 1.10 | 1.10 | 1.10 | 1.10 | 1.10 |
| Pigment | 0.22 | 0.66 | 0.26 | 0.22 | 0.26 |
| Magnesium stearate | 2.20 | 2.20 | 2.20 | 2.20 | 2.20 |
| Ac-Di-Sol | 4.40 | 4.40 | 4.40 | 4.40 | 4.40 |

| | Strength (mg) | | | |
|---|---|---|---|---|
| Component | 5 | 6 | 7.5 | 10 |
| Clathrate of warfarin | 5.46 | 6.55 | 8.18 | 10.91 |
| sodium salt | | | | |
| Water | 11.15 | 17.60 | 17.60 | 17.60 |
| Lactose | 140.62 | 138.43 | 137.99 | 135.39 |
| Avicel | 66.00 | 66.00 | 66.00 | 66.00 |
| Aerosil | 1.10 | 1.10 | 1.10 | 1.10 |
| Pigment | 0.22 | 1.32 | 0.13 | — |
| Magnesium stearate | 2.20 | 2.20 | 2.20 | 2.20 |
| Ac-Di-Sol | 4.40 | 4.40 | 4.40 | 4.40 |

In the preparation of the said tablets, a weighed amount of clathrate of warfarin sodium salt is dissolved in the given amount of water. Lactose, avicel, aerosil and pigment are mixed in a low-speed mixer, whereupon this mixture is sprayed upon with the above-prepared solution of clathrate of warfarin sodium salt in water. The obtained mixture is then dried to 2 to 4% by weight humidity, mixed with magnesium stearate and Ac-Di-Sol in a low-speed mixer and the obtained mixture is pressed into 200 mg tablets.

### Example 2

This example describes determination of content uniformity in 20 tablets of 1 to 10 mg strength, prepared by the procedure described in Example 1. The strengths given represent the lowest and the highest strengths used in the clinical practise, defining thus the whole range of dosage forms used in the clinical practise. The results obtained, documenting the high degree of content uniformity of tablets prepared according to the invention, are given in Table 2 below.

TABLE 2

| | Content | | | Limit RSD |
|---|---|---|---|---|
| | (limit: | Range | | according to |
| | 95-105%) | (limit: 85-115%) | RSD | Bergum |
| Warfarin 1 mg | 102% | 98-107% | 2.09% | 3.45% |
| Warfarin 10 mg | 99.4% | 97-102% | 1.27% | 3.90% |

1. The method of producing dosage units of a solid drug form containing as the active substance warfarin sodium salt in an amount of 1 to 10 mg and having high a degree of content uniformity meeting Bergum criterion, wherein a required amount of an aqueous solution of warfarin sodium salt and/or its clathrate which optionally contains in the dissolved state one of pharmaceutically acceptable excipients co-forming the solid drug form to be prepared but not all the pharmaceutically acceptable excipients co-forming the solid drug form to be prepared, is brought into contact with solid particles of at least two pharmaceutically acceptable excipients co-forming the solid drug form to be prepared, whereupon optionally the particles are dried and optionally mixed with the required amount of solid particles of the remaining pharmaceutically acceptable excipients co-forming the solid drug form to be prepared, and the thus-obtained particulate mixture is formulated into dosage units of the solid drug form.

2. The method according to claim 1, wherein the bringing into contact of an aqueous solution of warfarin sodium salt and/or its clathrate with solid particles of at least one pharmaceutically acceptable excipient is performed by spraying this solution onto these solid particles.

3. The method according to claim 1, wherein the aqueous solution of warfarin sodium salt and/or its clathrate contains 1 to 50% by weight of warfarin sodium salt and/or its clathrate, based on the weight of the solution.

4. The method according to claim 1, wherein the aqueous solution of warfarin sodium salt and/or its clathrate contains, besides water, solely warfarin sodium salt and/or its clathrate.

5. The method according to claim 1, wherein the solid particles of at least one pharmaceutically acceptable excipient intended for bringing into contact with aqueous solution of warfarin sodium salt and/or its clathrate, have such particle distribution that the size of at least 90% of these particles is greater than 40 micrometers, the size of at most 10% of these particles is greater than 250 micrometers, and 100% of these particles are of a size not exceeding 300 micrometers.

6. The method according to claim 1, wherein the pharmaceutically acceptable ingredients are hydrophilic sugar.

7. The method according to claim 1, wherein the solid particles of at least one pharmaceutically acceptable excipient, intended for bringing into contact with aqueous solution of warfarin sodium salt and/or its clathrate, contain added solid particles of a pharmaceutically acceptable excipient of a specific surface area of at least 150 m².g⁻¹ in an amount of 0.1 to 2% by weight based on the total weight of solid particles of at least one pharmaceutically acceptable excipient and the said added ingredient intended for bringing into contact with aqueous solution of warfarin sodium salt and/or its clathrate, preferably added colloidal silicon oxide, in an amount of 0.5% by weight based on the total weight of solid particles of at least one pharmaceutically acceptable excipient and the said added ingredient intended for bringing into contact with aqueous solution of warfarin sodium salt and/or its clathrate.

8. The method according to claim 1, wherein the mixture of solid, optionally dried particles, obtained after spraying an aqueous solution of warfarin sodium salt and/or its clathrate onto solid particles of at least one pharmaceutically acceptable excipient, is mixed with at least one pharmaceutically acceptable lubricant such as preferably magnesium stearate, zinc stearate, aluminium stearate, colloidal silicon oxide, stearic acid, sodium stearyl fumarate, polyethylene glycol or sodium lauryl sulfate, used in an amount of 0.1 to 10% by weight based on the weight of the obtained mixture, preferably with magnesium stearate used in an amount of 1% by weight based on the weight of the mixture obtained.

9. The method according to claim 1, wherein the mixture of solid, optionally dried particles, obtained after spraying an aqueous solution of warfarin sodium salt and/or its clathrate onto solid particles of at least one pharmaceutically acceptable excipient, is mixed with at least one pharmaceutically acceptable disintegrant such as preferably ultraamylopectin, sodium salt of crosslinked carboxymethylcellulose or crosslinked polyvinylpyrrolidone, used in an amount of 1 to 7% by weight, based on the weight of the mixture obtained, preferably with sodium salt of crosslinked carboxymethylcellulose, used in an amount of 2% by weight based on the weight of the mixture obtained.

10. The method according to claim 1, wherein the obtained particulate mixture is formulated into dosage units of solid drug form by filling into capsules and/or sachets, and/or by pressing to tablets.

11. The method according to claim 2, wherein the aqueous solution of warfarin sodium salt and/or its clathrate contains 1 to 50% by weight of warfarin sodium salt and/or its clathrate, based on the weight of the solution.

12. The method according to claim 2, wherein the aqueous solution of warfarin sodium salt and/or its clathrate contains, besides water, solely warfarin sodium salt and/or its clathrate.

13. The method according to claim 3, wherein the aqueous solution of warfarin sodium salt and/or its clathrate contains, besides water, solely warfarin sodium salt and/or its clathrate.

14. The method according to claim 2, wherein the solid particles of at least one pharmaceutically acceptable excipient intended for bringing into contact with aqueous solution of warfarin sodium salt and/or its clathrate, have such particle distribution that the size of at least 90% of these particles is greater than 40 micrometers, the size of at most 10% of these particles is greater than 250 micrometers, and 100% of these particles are of a size not exceeding 300 micrometers.

15. The method according to claim 3, wherein the solid particles of at least one pharmaceutically acceptable excipient intended for bringing into contact with aqueous solution of warfarin sodium salt and/or its clathrate, have such particle distribution that the size of at least 90% of these particles is greater than 40 micrometers, the size of at most 10% of these particles is greater than 250 micrometers, and 100% of these particles are of a size not exceeding 300 micrometers.

16. The method according to claim 4, wherein the solid particles of at least one pharmaceutically acceptable excipient intended for bringing into contact with aqueous solution of warfarin sodium salt and/or its clathrate, have such particle distribution that the size of at least 90% of these particles is greater than 40 micrometers, the size of at most 10% of these particles is greater than 250 micrometers, and 100% of these particles are of a size not exceeding 300 micrometers.

17. The method according to claim 1, wherein the pharmaceutically acceptable ingredients are selected from at least one of the groups consisting of sucrose, sorbitol, mannitol, lactose, natural starch, modified starch, and cellulose.

18. The method according to claim 1, wherein the pharmaceutically acceptable ingredients are a mixture of lactose and microcrystalline cellulose in a weight ratio of 10:5 to 11:5.

19. The method according to claim 2, wherein the solid particles of at least one pharmaceutically acceptable excipient, intended for bringing into contact with aqueous solution of warfarin sodium salt and/or its clathrate, contain added solid particles of a pharmaceutically acceptable excipient of a specific surface area of at least 150 m².g⁻¹ in an amount of 0.1 to 2% by weight based on the total weight of solid particles of at least one pharmaceutically acceptable excipient and the said added ingredient intended for bringing into contact with aqueous solution of warfarin sodium salt and/or its clathrate, preferably added colloidal silicon oxide, in an amount of 0.5% by weight based on the total weight of solid particles of at least one pharmaceutically acceptable excipient and the said added ingredient intended for bringing into contact with aqueous solution of warfarin sodium salt and/or its clathrate.

20. The method according to claim 2, wherein the mixture of solid, optionally dried particles, obtained after spraying an aqueous solution of warfarin sodium salt and/or its clathrate onto solid particles of at least one pharmaceutically acceptable excipient, is mixed with at least one pharmaceutically acceptable lubricant such as preferably magnesium stearate, zinc stearate, aluminium stearate, colloidal silicon oxide, stearic acid, sodium stearyl fumarate, polyethylene glycol or sodium lauryl sulfate, used in an amount of 0.1 to 10% by weight based on the weight of the obtained mixture, preferably with magnesium stearate used in an amount of 1% by weight based on the weight of the mixture obtained.

21. The method according to claim 2, wherein the mixture of solid, optionally dried particles, obtained after spraying an aqueous solution of warfarin sodium salt and/or its clathrate onto solid particles of at least one pharmaceutically acceptable excipient, is mixed with at least one pharmaceutically acceptable disintegrant such as preferably ultraamylopectin, sodium salt of crosslinked carboxymethylcellulose or crosslinked polyvinylpyrrolidone, used in an amount of 1 to 7% by weight, based on the weight of the mixture obtained, preferably with sodium salt of crosslinked carboxymethylcellulose, used in an amount of 2% by weight based on the weight of the mixture obtained.
